(19) United States
(12) Patent Application Publication
(10) Pub. No.: US 2023/0285104 A1
(43) Pub. Date: Sep. 14, 2023

(54) ENDORECTAL COIL DEVICE HOLDER

(71) Applicant: The Board of Regents of The University of Texas System, Austin, TX (US)

(72) Inventor: Xiaosong Meng, Dallas, TX (US)

(21) Appl. No.: 18/177,268

(22) Filed: Mar. 2, 2023

Related U.S. Application Data
(60) Provisional application No. 63/318,552, filed on Mar. 10, 2022.

(51) Int. Cl. A61B 50/20; A61B 5/055; G01R 33/34

(52) U.S. Cl. CPC A61B 50/20; A61B 5/055; G01R 33/34; A61B 2090/034

(57) ABSTRACT

An endorectal coil device holder is provided. The holder includes a base operable to be coupled to a surface, an arm extending form the base, and a receiver pivotably coupled with the arm. The receiver forms a receptacle operable to receive a coil base of the endorectal coil.

### CROSS-REFERENCE TO RELATED APPLICATION

This application claims the benefit of U.S. Provisional Patent Application Ser. No. 63/318,552 filed on Mar. 10, 2022, the content of which is incorporated by reference in its entirety.

### BACKGROUND

### 1. Field

The present disclosure relates generally to systems and methods related to endorectal coils. In at least one example, the present disclosure relates to a holder operable to maintain the positioning of an endorectal coil device.

### 2. Discussion of Related Art

Endorectal coils are used during endorectal coil magnetic resonance imaging (MRI) procedures for imaging of cancers of the rectum, prostate, and anus. During endorectal coil MRI, MRI is used in conjunction with an endorectal coil placed in the rectum to obtain high quality images of the area surrounding the rectum. Endorectal coil MRI determines the extent of spread and local invasion of cancer in the prostate, rectum, and/or anus. The endorectal coil helps focus on the prostate and surrounding structures, and the increased signal from the endorectal coil can help provide improved image quality. The endorectal coil can also enable the radiologist to perform magnetic resonance (MR) spectroscopy, which can provide additional information on the chemical makeup of cells present in the prostate gland. The endorectal coil must maintain its position and orientation to function as desired.

### BRIEF SUMMARY

The present inventive concept provides a holder for an endorectal coil. The holder is operable to receive the endorectal coil and maintain the position and orientation of the endorectal coil during an endorectal coil MRI.

The aforementioned may be achieved in an aspect of the present inventive concept by providing a holder for an endorectal coil. The holder may include a base operable to be coupled to a surface, an arm extending from the base, and a receiver pivotably coupled with the arm. The receiver may form a receptacle operable to receive a coil base of the endorectal coil and maintain the position of the endorectal coil.

The receptacle may include two longitudinal walls and two side walls. Each of the two side walls may form notches, where one of the notches is operable to receive a coil arm extending from a first end of the coil base and another one of the notches is operable to receive a coil cable extending from a second end opposite the first end of the coil base. The two side walls may be operable to abut against the coil base to retain the coil base within the receptacle. At least one of the two longitudinal walls and the two side walls may include a protrusion operable to correspond with and be received in a notch in the coil base to prevent rotation of the coil base. The receptacle may include a protrusion operable to correspond with and be received in a notch in the coil base to prevent rotation of the coil base. The base may include an adhesive which permits the base to be removably coupled to the surface. The adhesive may include a micro-suction tape. The arm may be rotatable in relation to the base. The arm may be translatable along a longitudinal axis of the base.

The aforementioned may also be achieved in an aspect of the present inventive concept by providing a system including an endorectal coil and a holder operable to receive and maintain the position of the endorectal coil. The holder may include a base operable to be coupled to a surface, an arm extending from the base, and a receive pivotably coupled with the arm. The receiver may form a receptacle operable to receive a coil base of the endorectal coil.

The aforementioned may also be achieved in an aspect of the present inventive concept by providing a kit including a holder for an endorectal coil and a syringe holder operable to receive a syringe. The holder may include a base operable to be coupled to a surface, an arm extending from the base, and a receiver pivotably coupled with the arm. The receiver may form a receptacle operable to receive a coil base of the endorectal coil.

The foregoing is intended to be illustrative and is not meant in a limiting sense. Many features of the embodiments may be employed with or without reference to other features of any of the embodiments. Additional aspects, advantages, and/or utilities of the present inventive concept will be set forth in part in the description that follows and, in part, will be apparent from the description, or may be learned by practice of the present inventive concept.

### BRIEF DESCRIPTION OF THE DRAWINGS

The description will be more fully understood with reference to the following figures and data graphs, which are presented as various embodiments of the present inventive concept and should not be construed as a complete recitation of the scope of the present inventive concept, wherein:

FIG. 1A illustrates an example of a system with an endorectal coil and a holder which may be used in accordance with the present disclosure;

FIG. 1B illustrates a top perspective view of the system of FIG. 1A;

FIG. 2A illustrates a top perspective view of a holder for an endorectal coil;

FIG. 2B illustrates a side view of the holder of FIG. 2A;

FIG. 2C illustrates an exploded view of the holder of FIG. 2A;

FIG. 2D illustrates a holder in a different configuration;

FIG. 3A illustrates a receiver operable to receive the endorectal coil;

FIG. 3B illustrates a top view of the holder;

FIG. 4A illustrates a holder with a hydraulic base;

FIG. 4B illustrates the holder of FIG. 4A, where the arm is translated along a longitudinal axis of the base;

FIG. 5A illustrates a holder with a rack base;

FIG. 5B illustrates a front perspective view of the holder of FIG. 5A;

FIG. 5C illustrates an enlarged view of the base of the holder of FIG. 5A; and

FIG. 5D illustrates the holder of FIG. 5A with a wedge preventing rearward movement along the base.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims. Further, it should be understood that any one of the features of the present inventive concept may be used separately or in combination with other features. Other systems, methods, features, and advantages of the present inventive concept will be, or become, apparent to one with skill in the art upon examination of the figures and the detailed description. It is intended that all such additional systems, methods, features, and advantages be included within this description, be within the scope of the present inventive concept, and be protected by the accompanying claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. General Architecture

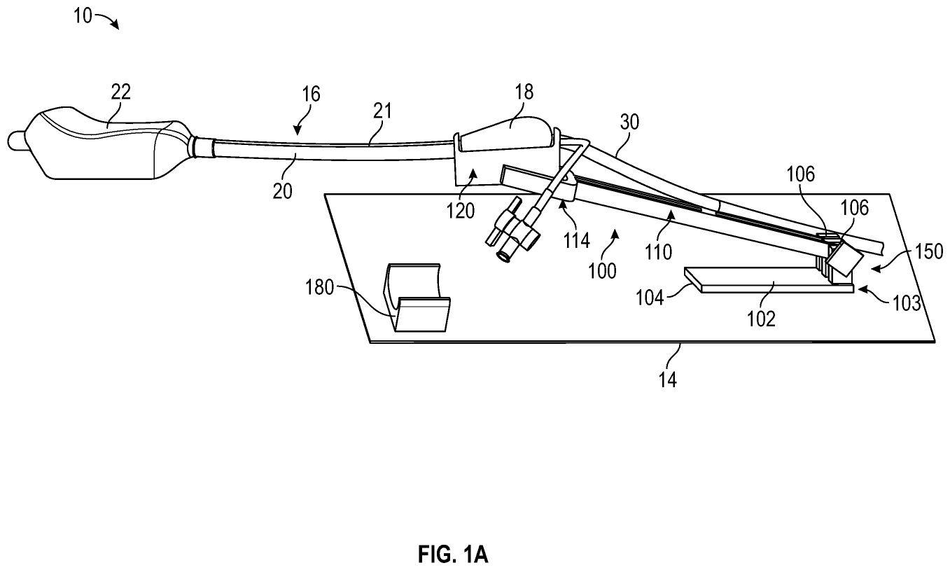

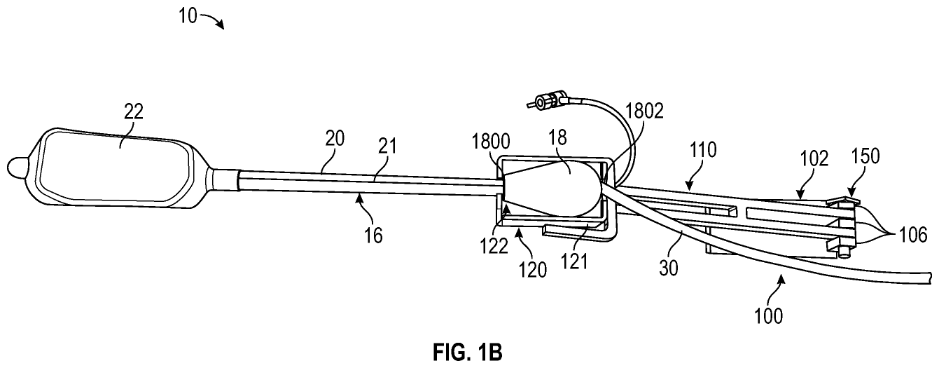

The disclosure now turns to FIGS. 1A and 1B, which illustrate an exemplary environment or system 10 for a holder 100 for an endorectal coil device 16, in which the present disclosure may be implemented. The system 10 can include an endorectal coil 16 and a holder 100 operable to receive and maintain the position of the endorectal coil 16. The endorectal coil 16 can be used during endorectal coil magnetic resonance imaging (MRI) procedures for imaging of cancers of the rectum, prostate, and anus. During endorectal coil MRI, MRI is used in conjunction with an endorectal coil 16 placed in the rectum to obtain high quality images of the area surrounding the rectum. Endorectal coil MRI determines the extent of spread and local invasion of cancer in the prostate, rectum, and/or anus. The endorectal coil 16 helps focus on the prostate and surrounding structures, and the increased signal from the endorectal coil 16 can help provide improved image quality. The endorectal coil 16 can also enable the radiologist to perform magnetic resonance (MR) spectroscopy, which can provide additional information on the chemical makeup of cells present in the prostate gland. The endorectal coil 16 must maintain its position and orientation to function as desired.

The endorectal coil 16 can include a coil cable 30, a coil base 18 coupled with the coil cable 30, a coil shaft 20 extending from the coil base 18 opposite the coil cable 30, and a balloon 22 surrounding the coil (not shown). The balloon 22 is coupled to the coil shaft 20 opposite the coil base 18. The balloon 22 is operable to be inflated to help keep the coil in place after being inserted into the rectum. The coil shaft 20 can include an alignment marker 21 to provide guidance that the endorectal coil 16 has the correct orientation. For example, as illustrated in FIGS. 1A and 1B, the alignment marker 21 can include a line across the coil shaft 20. The alignment marker 21 can be operable to provide visual assistance in achieving a desired positioning of the endorectal coil 16. The alignment marker 21, in the illustrated examples, must be oriented at the top of the coil shaft 20 for the endorectal coil 16 to be correctly oriented. Other alignment markers 21 such as arrows or ridges can be contemplated without deviating from the scope of the disclosure. The coil cable 30 is operable to couple the endorectal coil 16 to power and/or a controller (not shown). The system 10 can be disposed on a surface 14, such as a table or a tray.

In at least one example, the system 10 can include a syringe holder 180 which is operable to hold a syringe (not shown). For example, the syringe can be used to inflate and/or deflate the balloon 22. In at least one example, the syringe holder 180 can be provided along with the holder 100 as a kit for endorectal coil MRI procedures. The syringe holder 180 can have a recess that is operable to receive the syringe. The recess can have a cylindrical shape. In some examples, the recess can have a diameter between about 20 millimeters and about 30 millimeters. Alternately, the recess can have a diameter between about 23 millimeters and about 27 millimeters, alternately between about 24 millimeters and about 25 millimeters, alternately about 24.5 millimeters. In other examples, the syringe holder 180 can have any size and shape suitable to receive the syringe, for example via friction fit. In some examples, the syringe holder 180 can be coupled to the surface 14 to maintain the position of the syringe holder 180. As illustrated in FIG. 1A, the syringe holder 180 is disposed on the surface 14. For example, the syringe holder 180 can be coupled to the surface 14 by adhesion, by microsuction tape, or by any other suitable mechanism. By maintaining the position of the syringe holder 180 on the surface 14, the syringe holder 180 can be easily accessible and does not move as the syringe is placed in and/or on the syringe holder 180.

The positioning and orientation of the endorectal coil 16 is critical to the performance of the endorectal coil MRI. However, the endorectal coil may move in the patient's rectum which can affect the positioning and orientation of the endorectal coil 16. As illustrated in FIG. 1A, the holder 100 is operable to receive and maintain the position of the endorectal coil 16. Conventionally, the endorectal coil 16 is held in place with either tape to the patient's buttocks or with a sandbag. However, the conventional methods do not securely hold the endorectal coil 16 in place, allowing the endorectal coil 16 to either pull out or rotate, which can impact the performance of the procedure. The holder 100 disclosed herein is operable to securely hold the endorectal coil 16 substantially parallel to the MRI table so that the endorectal coil 16 does not rotate and does not move. In at least one example, as shown in FIG. 1A, the holder 100 is positioned on the system base or surface 20. In some examples, the holder 100 is operable to be positioned on the surface 14 as the table or tray or any other suitable surface 14.

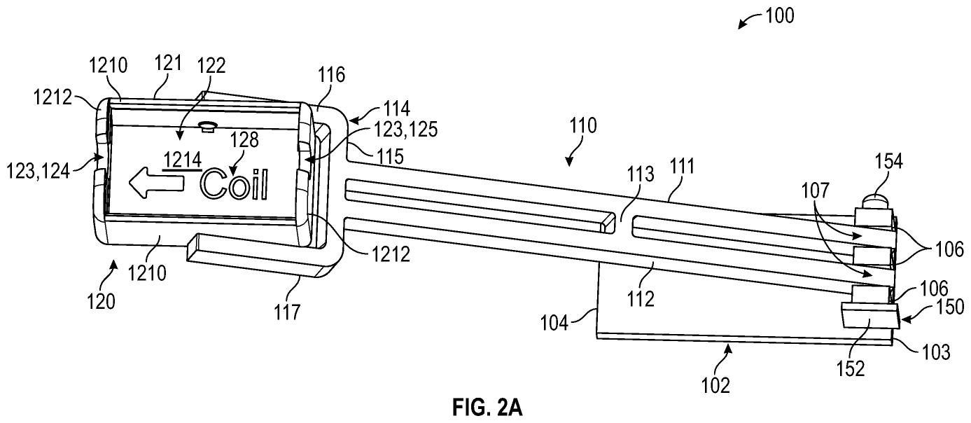

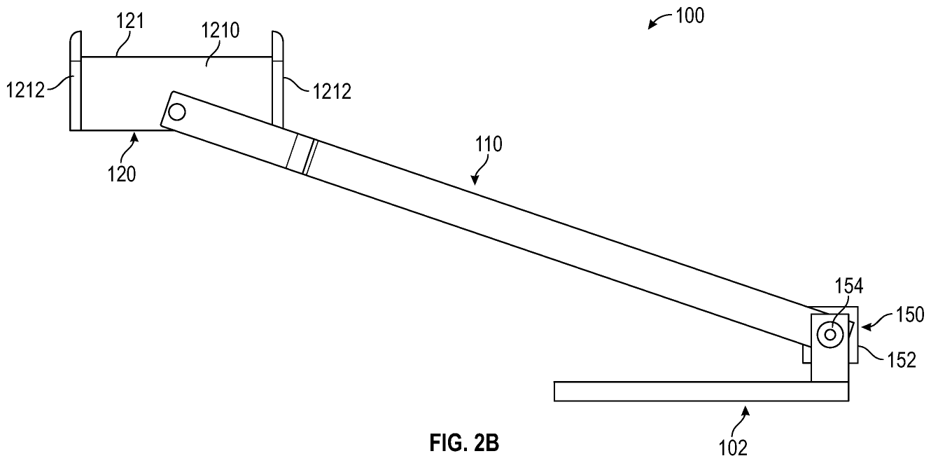

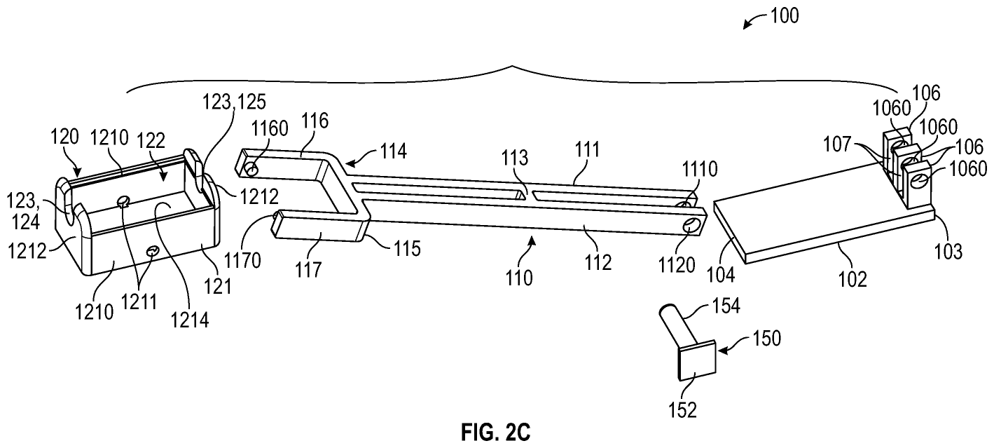

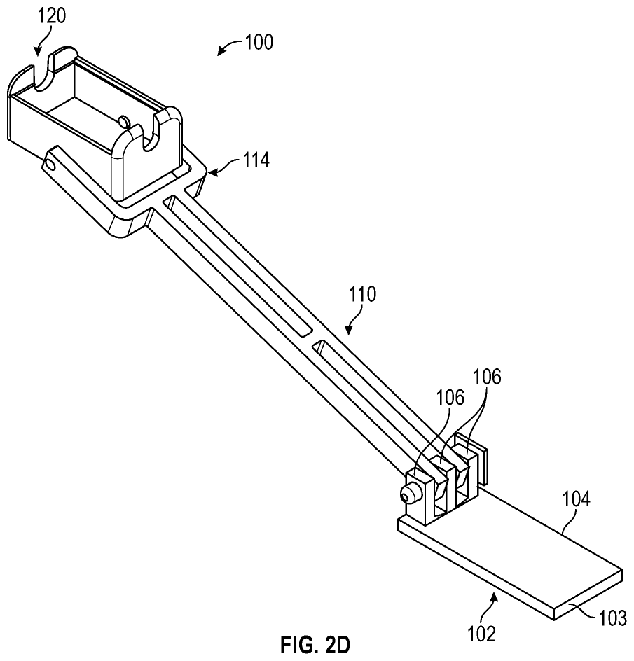

Referring to FIGS. 1A-2D, the holder 100 includes a base operable to be coupled to the surface 14, an arm 110 extending from the base 102, and a receiver 120 coupled with the arm 110.

In at least one example, the base 102 of the holder 100 is operable to be coupled to the surface 14. In some examples, the base 102 can include an adhesive which permits the base 102 to be removably coupled to the surface 14. For example, the adhesive can include a microsuction tape. In some examples, the base 102 can include a suction cup operable to removably couple the base 102 to the surface 14. In at least one example, when coupled with the surface 14, the base 102 is operable to be lifted vertically from the surface 14 to be removed. The base 102 may resist and/or prevent horizontal movement such as sliding to ensure the positioning of the holder 100 is maintained. In at least one example, the base 102 can have a length between about 70 millimeters and about 90 millimeters, alternately between about 75 millimeters and about 85 millimeters, alternately about 80 millimeters. The width of the base 102 can be between about 25 millimeters and about 45 millimeters, alternately between about 30 millimeters and about 40 millimeters, alternately about 35 millimeters. The size of the base 102 is configured to provide sufficient surface area for coupling with the surface 14. For example, with microsuction tape, the base 102 needs to have sufficient size so that the microsuction tape can hold on to the surface 14 without lifting off the surface during the procedure. Accordingly, the base 102 must be sufficiently coupled with the surface 14 to prevent undesired movement of the holder 100 and consequently the endorectal coil 16.

The patient's body may push against the endorectal coil 16 to eject the endorectal coil 16 from the rectum, so the holder 100 must resist that force to prevent the endorectal coil 16 from being pulled/pushed out of the rectum and/or rotate, which can impact the performance of the procedure. The base 102, as illustrated herein, is substantially rectangular in shape, as much of the force enacted upon the endorectal coil 16 and the holder 100 is along the longitudinal axis down the endorectal coil 16 and the arm 110 of the holder 100. However, in other examples, the base 102 can have an oval shape, a circular shape, a triangular shape, or any other suitable shape.

The base 102 can be coupled with the arm 110. In at least one example, the arm 110 can have a length between about 15 centimeters and about 26 centimeters. Alternately, the length of the arm 110 can be between about 18 centimeters and about 23 centimeters, alternately between about 20 centimeters and about 22 centimeters, alternately about 21 centimeters. The width of arm 110 can be between about 3 centimeters and about 9 centimeters. Alternately, the width of the arm 110 can be between about 5 centimeters and about 7 centimeters, alternately about 6 centimeters. The arm 110 has a length long enough so that the endorectal coil 16 can be inserted into the patient's rectum, and the arm 110 has a width wide enough so that the arm 110 does not twist and has sufficient strength to hold the endorectal coil 16 in place.

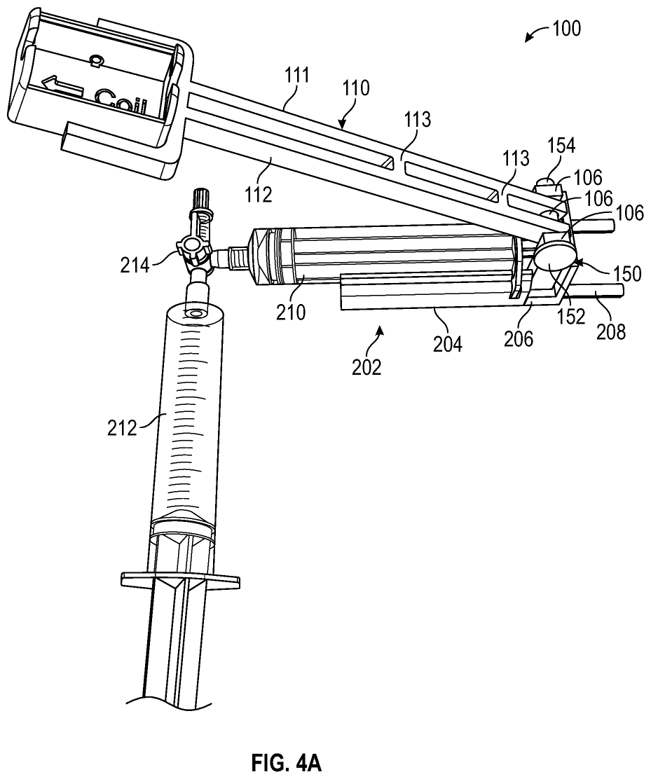

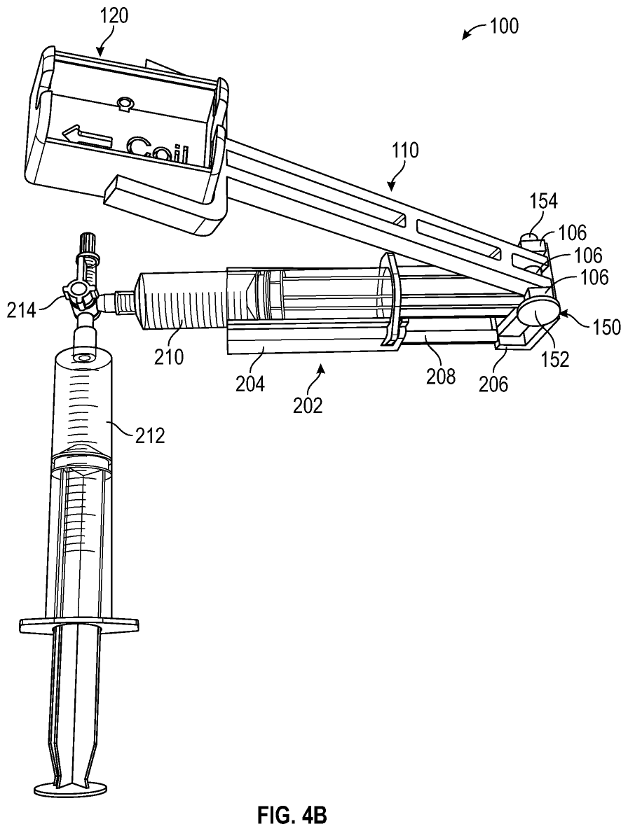

To achieve the desired stability and strength while minimizing weight and/or cost of manufacturing the holder 100, as illustrated, the arm 110 can include two longitudinal beams 111, 112 extending along a longitudinal axis of the arm 110 and at least one cross-beam 113 connecting the two longitudinal beams. In at least one example, the two longitudinal beams 111, 112 can be substantially parallel to one another. In some examples, the two longitudinal beams 111, 112 can be at an angle in relation to one another. In at least one example, as illustrated in FIGS. 2A-2D, the arm 110 can include one cross-beam 113. In some examples, as illustrated in FIGS. 4A-4B, the arm 110 can include two cross-beams 113 to further provide rigidity in the arm 110 and/or prevent rotation or twisting of the arm 110. In other examples, more than two cross-beams 113 can be included without deviating from the scope of the present disclosure.

In at least one example, to allow for adjustment of the holder 100 to accommodate different patients, the arm 110 can be rotatable in relation to the base 102. In some examples, the arm 110 can be immobile in relation to the base 102. In some examples, the arm 110 may be rotatable in relation to the base 102 but can be subject to sufficient friction or stopping force to prevent undesired movement unless a threshold force is enacted upon the arm 110. In some examples, the arm 110 may be rotatable in relation to the base 102 and be locked in the desired position.

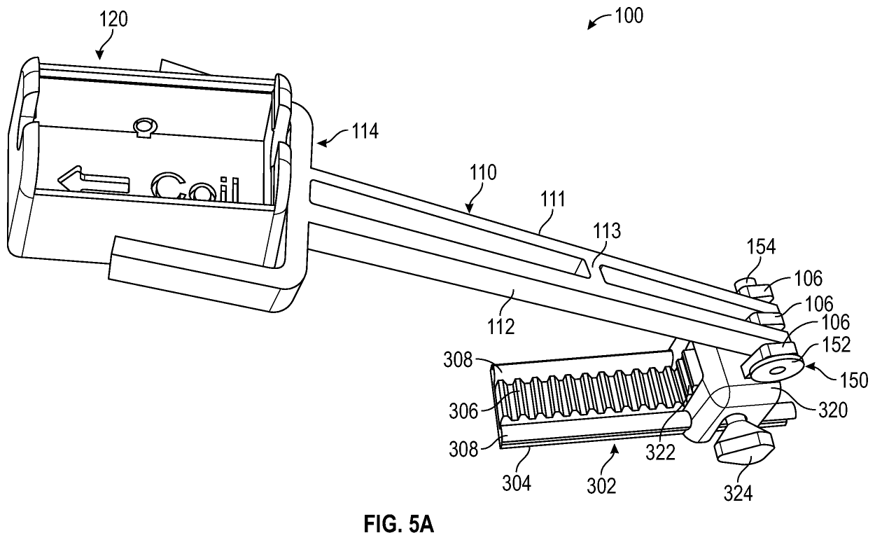

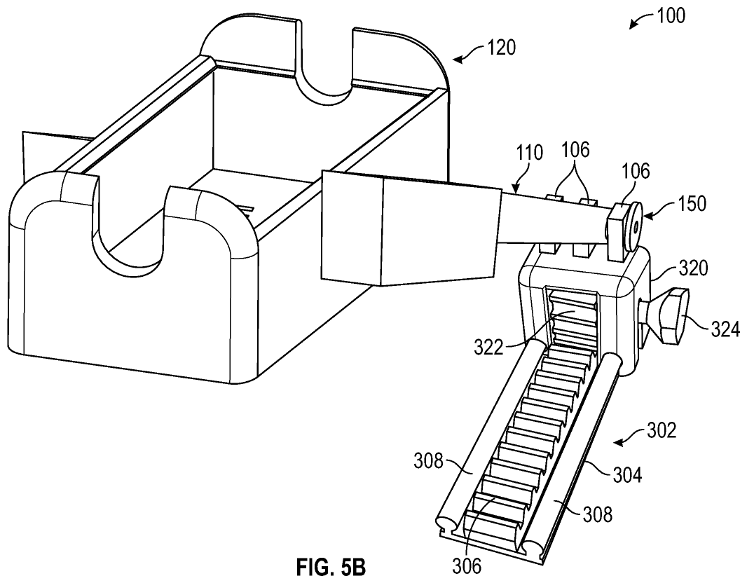

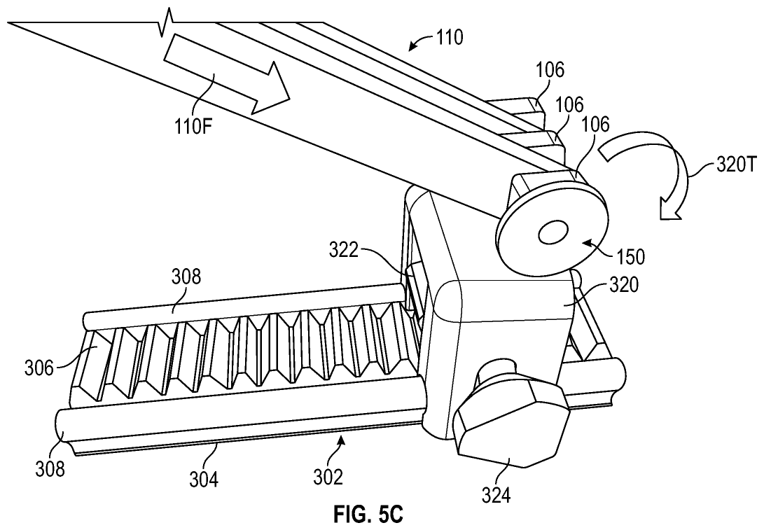

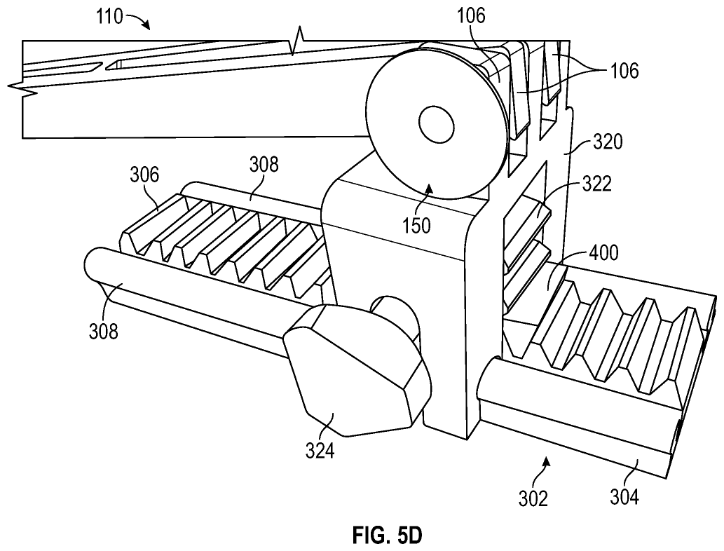

In at least one example, as illustrated herein, the arm 110 may be coupled with the base 102 with a pin 150. The pin 150 can include a head 152 and a shaft 154 extending from the head 152. The base 102 can include one or more projections 106. As shown in FIG. 2C, the projections 106 form holes 1060, and the longitudinal beams 111, 112 form holes 1110, 1120. The holes 1060 of the projections 106 can be aligned with the holes 1110, 1120 of the arm 110. When the arm 110 is aligned with the projections 106, the shaft 154 of the pin 150 can be inserted through the holes 1060 of the projections 106 and the holes 1110, 1120 of the arm 110. The head 152 of the pin 150 prevents the pin 150 from sliding all the way through the arm 110 and the projections 106. In at least one example, as illustrated in FIGS. 2A-2D, the head 152 of the pin 150 can have substantially a rectangular shape. In some examples, as illustrated in FIGS. 4A-5D where the arm 110 is translatable along the base 102, the head 152 of the pin 150 can have substantially a circular shape to prevent collision with the base 102. In some examples, a cap (not shown) can be coupled to the shaft 154 of the pin 150 opposite the head 152 to secure the pin 150 in place.

As illustrated in FIGS. 2A-2D, the projections 106 can be substantially rectangular, but the projections 106 can be any suitable shape without deviating from the scope of the disclosure. In at least one example, the base 102 includes one projection 106, and the longitudinal beams 111, 112 of the arm 110 are positioned on either side of the projection 106. In at least one example, the base 102 can include a plurality of projections 106 which form gaps 107 in between the projections 106. The gaps 107 are operable to receive the arm 110. As illustrated herein, the base 102 includes three projections 106 to form two gaps 107 such that the two longitudinal beams 111, 112 can be received in the two gaps 107.

In at least one example, the arm 110 can be coupled to the base 110 proximate the rear end 104 of the base 102. As illustrated in FIGS. 1A-2C, the holder 100 may be configured such that the arm 110 extends from the rear end 104 away from a front end 103 of the base 102, the front end 103 being opposite the rear end 104. In some examples, as illustrated in FIG. 2D, the holder 100 may be configured such that the arm 110 extends from the rear end 104 towards the front end 103 of the base 102. In some examples, the arm 110 can be rotated about the base 102 to move between the two configurations as needed. Either of the two configurations may be used depending on how far away the patient is from the base 102 of the holder 100.

The receiver 120 is coupled with the arm 110 opposite the base 102. As shown in FIGS. 1A-1B, the receiver 102 is operable to receive at least a portion of the endorectal coil 16 and maintain the position of the endorectal coil 16. For example, the receiver 102 can be operable to receive the coil base 18. The receiver 102 has a frame 121 which forms a receptacle 122 which is operable to receive the endorectal coil 16.

In at least one example, the receiver 120 is pivotably coupled with the arm 110. The arm 110 can include a bracket 114 operable to receive the receiver 120. The bracket 114 can include a bottom portion 115 and two branches 116, 117 extending from opposing ends of the bottom portion 115. The bottom portion 115 can be coupled to and/or extending from the longitudinal beams 111, 112. The bottom portion 115 can extend substantially perpendicular to the longitudinal beams 111, 112, and the two branches 116, 117 can extend substantially perpendicular to the bottom portion 115. As can be seen in FIG. 2C, the branch 116 can include a protrusion 1160 therefrom, and the branch 117 can include a protrusion 1170 extending therefrom. In at least one example, as illustrated herein, the protrusion 1160 can extend from the branch 116 towards the branch 117, and the protrusion 1170 can extend from the branch 117 towards the branch 116. The receiver 120 includes two holes or recesses 1211 formed in opposing sides of the frame 121 corresponding to the positions of the protrusions 1160, 1170. The protrusions 1160, 1170 can be inserted into the corresponding hole or recess 1211. Accordingly, the receiver 120 is received in the bracket 114 of the arm 110 and can be pivoted about the protrusions 1160, 1170. As the receiver 120 is pivotably coupled with the arm 110, the receiver 102 and the endorectal coil 16 can be pivoted to ensure the proper orientation and positioning of the endorectal coil 16.

As mentioned above, the receiver 120 can have a frame 121 which forms a receptacle 122. The receptacle 122 is shaped to receive the endorectal coil 16. For example, the receptacle 122 can be substantially rectangular in shape. The receptacle 122 can include two longitudinal walls 1210 and two side walls 1212. The two side walls 1212 can connect the two longitudinal walls 1210 on opposing ends of the longitudinal walls 1210. In at least one example, the two longitudinal walls 1210 can extend parallel to one another, and the two side walls 1212 can extend parallel to one another but perpendicular to the longitudinal walls 1210. In at least one example, the receptacle 122 can include a floor 1214 coupled to each of the longitudinal walls 1210 and the side walls 1212.

In some examples, the receptacle 122 can have a width between about 21 millimeters and about 41 millimeters, alternately between about 26 millimeters and about 36 millimeters, alternately about 31 millimeters. The receptacle 122 can have a length between about 42 millimeters and about 62 millimeters, alternately between about 47 millimeters and about 57 millimeters, alternately about 52 millimeters. Accordingly, the receptacle 122 is operable to securely receive the endorectal coil 16 to maintain the position of the endorectal coil 16. In at least one example, the receptacle 122 can include notches 123 which are operable to receive the coil cable 30 and the coil shaft 20 while the coil base 18 is received in the receptacle 122. For example, each of the two side walls 1212 can form notches 123. A first notch 125 in one of the side walls 1212 can be operable to receive the coil cable 30. A second notch 124 in the other of the two side walls 1212 can be operable to receive the coil shaft 20. As can be seen in FIG. 1B, due to the notches 123, the coil base 18 can sit deeper and/or fully within the receptacle 122. A first end 1802 of the coil base 18 from which the coil cable 30 extends is wider than the first notch 125, so at least a portion of the first end 1802 of the coil base 18 abuts against the side wall 1212. A second end 1800 of the coil base 18 from which the coil shaft 20 extends is wider than the second notch 124, so at least a portion of the second end 1800 of the coil base 18 abuts against the side wall 1212. Accordingly, the two side walls 1212 are operable to abut against the coil base 18 to retain the coil base within the receptacle 122 and prevent forward and backward movement of the coil base 18 and subsequently the endorectal coil 16. The notches 123 receiving the coil cable 30 and the coil shaft 20 prevent lateral movement of the coil base 18 and subsequently the endorectal coil 16. Also, the longitudinal walls 1210 can prevent lateral movement of the coil base 18 and subsequently the endorectal coil 16. It is contemplated that the size and shape of the receptacle 122 can be different, so long as the receptacle 122 can securely receive the endorectal coil 16 without undesired movement of the endorectal coil 16. In some examples, coupling mechanisms may be included to couple the endorectal coil 16 with the receiver 120 within the receptacle 122. For example, adhesive may be included on the inner surfaces of the receptacle 122 that are operable to couple with the endorectal coil 16. In some examples, extensions may protrude outward from the receptacle 122 to abut against the endorectal coil 16.

As shown in FIG. 2A, the receiver 120 can include an orientation marker 128. The orientation marker 128 can indicate to a user how the endorectal coil 16 should be placed in the holder 100. For example, as illustrated in FIG. 2A, the orientation marker 128 can include the word “COIL” and an arrow pointing to the direction that the balloon 22 should be after the endorectal base 18 is placed in the receptacle 122. Other styles of orientation markers 128 can be contemplated without deviating from the scope of the disclosure, so long as the orientation marker 128 indicates the desired orientation of the endorectal coil 16 in relation to the holder 100. As illustrated in FIG. 2A, the orientation marker 128 is on the floor 1214 of the receptacle 122. It is contemplated that the orientation marker 128 can be placed in other positions, for example the longitudinal walls 1210, without deviating from the scope of the disclosure.

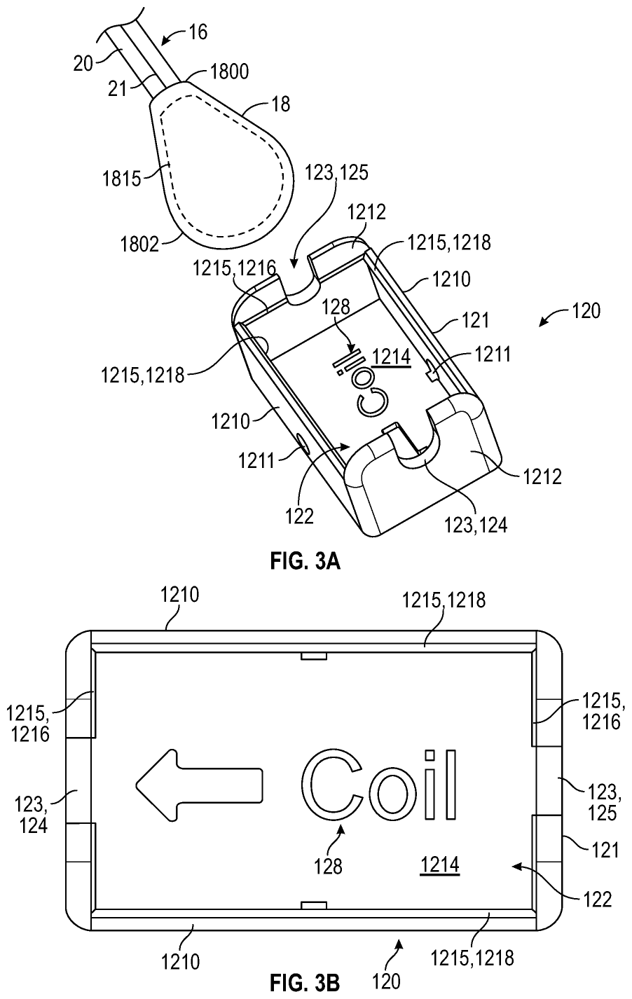

As illustrated in FIGS. 3A and 3B, the receiver 120 may include further components to secure the position of the endorectal coil 16 within the receptacle 122 and to maintain the orientation of the endorectal coil 16 by preventing rotation of the endorectal coil 16. As can be seen in FIG. 3A, the endorectal coil 16 may include a notch 1815 in the coil base 18. In at least one example, the notch 1815 can be formed around the entire coil base 18. In some examples, the notch 1815 can include one notch 1815 or a plurality of notches 1815 in different places on the coil base 18. The receptacle 122 can include at least one protrusion 1215 operable to correspond with and be received in a notch 1815 in the coil base 18 to prevent rotation of the coil base 18. In some examples, at least one of the two longitudinal walls 1210 and the two side walls 1212 can include a protrusion 1215 that corresponds with and is operable to be received in the notch 1815 in the coil base 18. For example, as illustrated in FIGS. 3A and 3B, the two longitudinal walls 1210 each include a notch 1218, and the two side walls 1212 each include a notch 1216. These notches 1815 are included in conventional endorectal coils 16. Correspondingly, the receptacle 122 can be retrofit to the endorectal coil 16 and include protrusion(s) 1215 that correspond with and are operable to be received by the notch(es) 1815 on the coil base 18 of the endorectal coil 16. The protrusion(s) 1215 being received in the notch(es) 1815 prevents the endorectal coil 16 from rotating. Accordingly, the positioning and orientation of the endorectal coil 16 is maintained.

As the receiver 120 can pivot in relation to the arm 110 and the arm 110 can rotate in relation to the base 102, the endorectal coil 16 can be positioned to be parallel to the table that the patient is on and is securely held in place during the procedure.

In at least one example, the receiver 120 can be removable from the arm 110. In some examples, the receiver 120 may be disposable and replaced for each procedure.

FIGS. 4A-5D illustrate alternative bases 202, 302 to increase maneuverability of the holder 100 to better position the endorectal coil 16 as each patient may require a different position. The bases 202, 302 as illustrated in FIGS. 4A-5D provide different configurations to provide a base 202, 302 so that the arm 110 can be translatable along a longitudinal axis of the base 202, 302. This can assist in adjusting the positioning of the endorectal coil 16 for the patient without having to lift, move, and place the base 102 of the holder 100 until the distance between the holder 100 and the patient is as desired. As illustrated in FIGS. 4A-5D, each of the different bases 202, 302 are configured such that the arm 110 and the receiver 120 as discussed above can fit with each base 202, 302. Accordingly, as discussed herein, the bases 102, 202, 302 discussed herein are interchangeable, so the bases 102, 202, 302 can be swapped out as needed depending on the patient. To change the base 102, 202, 302, the pin 150 can be removed, the arm 110 can then be separated from the protrusions 106, and a new base 102, 202, 302 can be put into place and coupled to the arm 110 via the pin 150. Other mechanisms translate the arm 110 along the base 202, 302 can be contemplated without deviating from the scope of the disclosure.

FIGS. 4A-4B illustrate a holder 100 with a hydraulic base 202. The hydraulic base 202 includes a support 204 and a pedestal 206. The support 204 and the pedestal 206 are coupled to one another such that the pedestal 206 can translate along a longitudinal axis in relation to the support 204. The hydraulic base 202 can include a pump 212 and a piston 210 that are operable to move the pedestal 206 in relation to the support 204. As the pump 212 either pushes fluid into the piston 210 or pulls fluid from the piston 210, the piston 210 translates, causing either the support 204 or the pedestal 206 to move in relation to one another. In the example illustrated in FIGS. 4A and 4B, the pump 212 can be hydraulicly coupled to the piston 210 via a hydraulic coupler 214. In at least one example, the hydraulic coupler 214 can include one or more valves. In some examples, the hydraulic coupler 214 can include one or more tubing to provide some separation from the components of the holder 100. In at least one example, as illustrated in FIGS. 4A and 4B, the pump 212 and/or the piston 210 can include a syringe.

In some examples, as illustrated in FIGS. 4A and 4B, as the piston 210 moves, the pedestal 206 translates back and forth along the longitudinal axis of the hydraulic base 202 by sliding away from and towards the support 204. As the arm 110 is coupled to the hydraulic base 202 via the protrusions 106 on the pedestal 206, the arm 110 translates along the longitudinal axis along with the pedestal 206. In some examples, the arm 110 can be coupled to the hydraulic base 202 via the support 204, and the support 204 may translate instead of the pedestal 206.

In at least one example, as illustrated in FIGS. 4A and 4B, the hydraulic base 202 can include one or more guides 208. In some examples, the guides 208 can be coupled to and/or extend from the support 204, and the pedestal 206 is movably coupled or seated on the guides 208. For example, the guides 208 can serve as railing along which the pedestal 206 slides or translates along the longitudinal axis. In some examples, the guides 208 can be coupled to and/or extend from the pedestal 206, and the guides are received in the support 204. For example, the support 204 may remain in place and includes channels to receive the guides 208. As the pedestal 206 translates along the longitudinal axis due to the movement of the piston 210, the guides 208 remain within the channels in the support 204 but translate with the pedestal 206. The guides 208 can maintain aligned connection between the support 204 and the pedestal 206. For example, the guides 208 prevent lateral rotation of the pedestal 206 and subsequently the arm 110.

FIGS. 5A-5D illustrate a holder 100 with a rack base 302. The rack base 302 can include a support 304 and one or more guides 308 that is movably coupled to a cage 320. For example, the guides 308 can serve as railing along which the cage 320 slides or translates along the longitudinal axis. The cage 320 can include the protrusions 106 which are used to couple to the arm 110. The support 304 can include a guide rack 306 which can include a plurality of crests and valleys. The rack base 302 can include a knob 324 which, when turned, is operable to move the cage 320 along the guides 308. For example, when the knob 324 is turned, a cage rack 322 is turned. As the cage rack 322 turns, the crests on the cage rack 324 are moved into a corresponding valley on the guide rack 306, and the crests on the guide rack 306 are moved into a corresponding valley on the cage rack 322. This moves the cage rack 322 and the cage 320 longitudinally along the guide rack 306 and the support 304.

In at least one example, as illustrated in FIG. 5C, after the position of the cage 320 and correspondingly the arm 110 of the holder 100 is set, force 110F of the arm 110 is enacted on the cage 320. The force 110F can be imparted on the holder 100 by the endorectal coil 16 in the patient. As the projections 106 are disposed proximate the rear of the cage 320, the force 110F places torque 320T on the cage 320 and prevents the cage 320 from undesired movement backwards. In at least one example, as illustrated in FIG. 5D, a wedge 400 can be placed on the guide rack 306 to mechanically prevent the cage 320 from undesired movement. For example, to prevent backward movement, a wedge 400 can be placed behind the cage 320. To prevent forward movement, a wedge 400 can be placed in front of the cage 320.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

What is claimed is:

1. An endorectal coil holder comprising:
a base operable to be coupled to a surface;
an arm extending from the base; and
a receiver pivotably coupled with the arm, the receiver forming a receptacle operable to receive a coil base of the endorectal coil and maintain the position of the endorectal coil.

2. The holder of claim 1, wherein the receptacle includes two longitudinal walls and two side walls.

3. The holder of claim 2, wherein each of the two side walls forms notches, wherein one of the notches is operable to receive a coil arm extending from a first end of the coil base and another one of the notches is operable to receive a coil cable extending from a second end opposite the first end of the coil base.

4. The holder of claim 2, wherein the two side walls are operable to abut against the coil base to retain the coil base within the receptacle.

5. The holder of claim 2, wherein at least one of the two longitudinal walls and the two side walls include a protrusion operable to correspond with and be received in a notch in the coil base to prevent rotation of the coil base.

6. The holder of claim 1, wherein the receptacle includes a protrusion operable to correspond with and be received in a notch in the coil base to prevent rotation of the coil base.

7. The holder of claim 1, wherein the base includes an adhesive which permits the base to be removably coupled to the surface.

8. The holder of claim 6, wherein the adhesive includes a micro-suction tape.

9. The holder of claim 1, wherein the arm is rotatable in relation to the base.

10. The holder of claim 1, wherein the arm is translatable along a longitudinal axis of the base.

11. The holder of claim 1, wherein the receiver is pivotable in relation to the arm.

12. A system comprising:
an endorectal coil; and
a holder operable to receive and maintain the position of the endorectal coil, the holder including:
a base operable to be coupled to a surface;
an arm extending from the base; and
a receiver pivotably coupled with the arm, the receiver forming a receptacle operable to receive a coil base of the endorectal coil.

13. The system of claim 12, wherein the endorectal coil includes an alignment marker operable to provide visual assistance in achieving a desired positioning of the endorectal coil.

14. The system of claim 12, wherein the receptacle includes two longitudinal walls and two side walls.

15. The holder of claim 14, wherein each of the two side walls forms notches, wherein one of the notches is operable to receive a coil arm extending from a first end of the coil base and another one of the notches is operable to receive a coil cable extending from a second end opposite the first end of the coil base.

16. The holder of claim 14, wherein the two side walls are operable to abut against the coil base to retain the coil base within the receptacle.

17. The holder of claim 14, wherein at least one of the two longitudinal walls and the two side walls include a protrusion operable to correspond with and be received in a notch in the coil base to prevent rotation of the coil base.

18. The holder of claim 14, wherein the receptacle includes a protrusion operable to correspond with and be received in a notch in the coil base to prevent rotation of the coil base.

19. The holder of claim 14, wherein the base includes an adhesive which permits the base to be removably coupled to the surface.

20. The holder of claim 1, wherein the arm is translatable along a longitudinal axis of the base.
